(19) United States
(12) Patent Application Publication
(10) Pub. No.: US 2015/0224275 A1
(43) Pub. Date: Aug. 13, 2015

(54) CUSTOMISATION OR ADJUSTMENT OF PATIENT INTERFACES

(71) Applicants: Sander Theodoor Pastoor, Utrecht (NL); Thomas Vollmer, Aachen (DE); Peter Chi Fai Ho, Pittsburgh, PA (US); Christoph Dobrusskin, Eindhoven (NL)

(72) Inventors: Sander Theodoor Pastoor, Utrecht (NL); Thomas Vollmer, Aachen (DE); Peter Chi Fai Ho, Pittsburgh, PA (US); Christoph Dobrusskin, Eindhoven (NL)

(21) Appl. No.: 14/419,766

(22) PCT Filed: Jul. 29, 2013
(86) PCT No.: PCT/IB2013/056191

§ 371 Date: Feb. 5, 2015

Related U.S. Application Data
(60) Provisional application No. 61/681,175, filed on Aug. 9, 2012.

(51) Int. Cl. A61M 16/06

(52) U.S. Cl. CPC A61M 16/0622; A61M 16/0633; A61M 2205/13

(57) ABSTRACT

A sensor device in the form of a patient interface has a sensor arrangement for determining a degree of fitting of a contact surface to the patient. This enables design parameters for a customised patient interface to be determined or else fitting adjustments to be made, based on how well the customisation sensor device fits the patient.

### FIELD OF THE INVENTION

The present invention relates to patient interfaces for transporting a gas to and/or from an airway of a user, and relates in particular to the customisation or adjustment of the patient interface to a particular user.

### BACKGROUND OF THE INVENTION

There are numerous situations where it is necessary or desirable to deliver a flow of breathing gas non-invasively to the airway of a patient, i.e. without inserting a tube into the airway of the patient or surgically inserting a tracheal tube in their oesophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnoea syndrome, in particular, obstructive sleep apnoea (OSA).

Non-invasive ventilation and pressure support therapies involve the placement of a patient interface assembly, including a patient interface in the form of a mask component, on the face of a patient. The mask component may be, without limitation, a nasal mask that covers the patient's nose, a nasal pillow/cushion having nasal prongs that are received within the patient's nostrils, a nasal/oral mask that covers the nose and mouth, or a full face mask that covers the patient's face. The patient interface interfaces between the ventilator or pressure support device and the airway of the patient, so that a flow of breathing gas can be delivered from the pressure/flow generating device to the airway of the patient.

Such assemblies are typically maintained on the face of a patient by headgear having one or more straps adapted to fit over/around the patient's head.

FIG. 1 shows a typical system to provide respiratory therapy to a patient. This system will be referred to in the description as a “patient interface assembly”.

The assembly 2 includes a pressure generating device 4, a delivery conduit 16 coupled to an elbow connector 18, and a patient interface 10. The pressure generating device 4 is structured to generate a flow of breathing gas and may include, without limitation, ventilators, constant pressure support devices (such as a continuous positive airway pressure device, or CPAP device), variable pressure devices, and auto-titration pressure support devices.

Delivery conduit 16 communicates the flow of breathing gas from pressure generating device 4 to patient interface 10 through the elbow connector 18. The delivery conduit 16, elbow connector 18 and patient interface 10 are often collectively referred to as a patient circuit.

The patient interface includes a mask 12 in the form of a shell 15 and cushion 14, which in the exemplary embodiment is nasal and oral mask. However, any type of mask, such as a nasal-only mask, a nasal pillow/cushion or a full face mask, which facilitates the delivery of the flow of breathing gas to the airway of a patient, may be used as mask. The cushion 14 is made of a soft, flexible material, such as, without limitation, silicone, an appropriately soft thermoplastic elastomer, a closed cell foam, or any combination of such materials.

An opening in the shell 15, to which elbow connector 18 is coupled, allows the flow of breathing gas from pressure generating device 4 to be communicated to an interior space defined by the shell 15 and cushion 14, and then to the airway of a patient.

The patient interface assembly also includes a headgear component 19, which in the illustrated embodiment is a two-point headgear. Headgear component 19 includes a first and a second strap 20, each of which is structured to be positioned on the side of the face of the patient above the patient's ear.

Headgear component 19 further includes a first and a second mask attachment element 22 to couple the end of one of the straps 20 to the respective side of mask 12.

A problem with this type of assembly is that the headgear force vectors necessary to achieve a robust and stable seal against the face of the patient can cut a straight line near the corners of a patient's eyes, which can be uncomfortable and distracting.

In order to avoid this, it is well known to include a forehead support to spread the required forces over a larger area. In this way, an additional cushion support on the forehead balances the forces put by the patient interface (the mask) around the nose or nose and mouth. Current masks have three to five sizes per mask type to cover the user population. Sizes are identified as Small (S), Medium (M), Large (L), Extra Large (XL), and Double Extra Large (XXL).

The variations in nose bridge height, nose width and the contour around the mouth (in case of full face masks) are spots where leaking can occur or where the seal is tightened too much causing too much pressure on local spots on the face. A perfect fitting mask will require a reduced force to seal well.

It is known that it would be desirable to customise each patient interface mask to the particular user. For example, a scan of the user's face has been proposed, from which a (virtual) mask model can be derived, and then used to create a customised mask. However, this scanning operation requires expensive equipment.

### SUMMARY OF THE INVENTION

According to the invention, there is provided a device and method as claimed in the independent claims.

In one aspect, the invention provides a sensor device comprising: a patient interface cushion having a contact surface for making contact with the face of the patient when the customisation sensor device is worn by the patient; and a sensor arrangement provided for determining parameters relating to the degree of fitting of the contact surface to the patient, thereby to enable design parameters to be obtained for a customised patient interface or to enable fitting adjustments of the patient interface to be made.

This device enables the degree of fitting of a patient interface to be detemrined, and this information can be used to change the patient interface design or else change the way it is fitted.

In a first set of examples, the sensor device is a customisation device. The device then enables parameters for a customised patient interface to be obtained in a simple, cost effective manner and in a way which provides a reliable customisation process. The parameter relating the degree of fitting can for example be a force, a pressure, or a physical displacement, caused by fitting the contact surface to the patient.

The use of this device enables a customized patient interface to be obtained by applying a real patient interface to the user, and this device can be thought of as a template device. By physically applying the device to the patient (rather than performing an optical scan, for example), skin deformation will take place so that the actual way the device fits will be taken into account. The personal variation is then measured based on this template device.

The load on the face can thus also be taken into account.

In another set of examples, the sensor device is part of a patient interface system and is to enable fitting adjustments of the patient interface to be made. The system can for example have a holding arrangement (for exampel a strap arrangement) for holding the patient interface in contact with the face of the patient and adjustment means for adjusting the holding arrangement, wherein the adjustment means is controlled to provide said fitting adjustments based on the sensor arrangement signals. The adjustment means can comprise manual adjustment means for control by the patient (this can be for large adjustments) and automatic adjustment means for control based on the sensor arrangment signals (this can be for fine adjustments).

The sensor arrangement is preferably provided in the vicinity of the contact surface, for determining a degree of fitting of the contact surface to the patient.

The patient interface can be for communicating a breathing gas to a patient. In the case of the customisation examples, the template device can thus be based on existing commercially available masks, so there can for example be a set of template devices corresponding to the typical range of mask sizes. The template devices have the integrated sensor arrangement to enable the individual facial contours and contact forces or pressures to be derived.

In this way, a set of customisation devices of different sizes can be provided. A set of patient interfaces of different sizes corresponding to the sizes of the customisation devices are then provided and the shape of the contact surface is adjustable.

The sensor arrangement can enable the shape of the contour of the seal cushion to the skin to be obtained. The seal cushion can comprise multiple parts, for example a first cushion part which prevents pressurized air from escaping, and a second cushion part which supports the mask arrangement on pressure insensitive parts of the user's face.

The patient interface can comprise a patient interface element (in the form of a mask part) and a forehead support, and the sensor arrangement can be for determining a degree of fitting of the contact surface of either one or both of the patient interface element and the forehead support.

A customisation system for a patient interface can comprise a customisation device of the invention and a processor for determining, from the sensor arrangement signals, the design parameters for a customised patient interface.

In another aspect, the invention provides a method of using a patient interface, comprising:
applying a sensor device to a patient, the sensor device comprising a patient interface having a contact surface for making contact with the face of the patient when the device is worn by the patient and a sensor arrangement for determining parameters relating to the degree of fitting of the contact surface to the patient;
from the parameters relating to the degree of fitting of the contact surface to the patient obtaining design parameters for a customised patient interface or making fitting adjustments of the patient interface.

In one set of examples, the method is for customising a patient interface, and the method comprises:
determining from the sensor arrangement signals design parameters for a customised patient interface; and
customising a patient interface using the design parameters.

In this case, the method can comprise selecting one customisation sensor device from a set of customisation sensor devices of different sizes, wherein the selected customisation device is the one applied to the patient; and
selecting one of a set of patient interfaces of different sizes, the one selected having a corresponding size to the selected customisation sensor device, and wherein the design parameters are used to customise the selected patient interface.

In another set of examples, the parameters relating to the degree of fitting is used to make fitting adjustments of the patient interface, wherein the patient interface is part of a patient interface system which further comprises a holding arrangement for holding the patient interface in contact with the face of the patient and adjustment means for adjusting the holding arrangement,
wherein the method comprises controlling the adjustment means to provide said fitting adjustments based on the sensor arrangement signals.

This approach provides automatic adjustment. Instead, the fitting adjustments can be enabled by providing an instruction to the patient, for example identifying that one or more straps of the strap arrangement are too tight or too loose.

In all cases, determining a degree of fitting can comprise:
using an optical fiber ring sensor (spatial continous sensing) or distributed optical fiber sensors (spatial localized sensing) to provide strain/contact-pressure/contact-force information at different positions around the contact surface; or
using an array of strain gauges around the contact surface.

The strain gauges can be mechanical or optical, or they can be mechanical with optical read out.

### BRIEF DESCRIPTION OF THE DRAWINGS

Examples of the invention will now be described in detail with reference to the accompanying drawings, in which:

FIG. 1 shows a known patient interface;

FIG. 2 shows a known patient interface as disclosed in U.S. 2010/0000542;

FIG. 3 shows a first example of device of the invention;

FIG. 4 shows in cross section the routing of optical fibers through the cushion;

FIG. 5 shows how a fiber Bragg grating arrangement is used to provide multiple strain measurements from a single fiber;

FIG. 6 shows a second example of device of the invention;

FIG. 7 shows an example of a customisable patient interface device which can customised using the measurement arrangement of the invention;

FIG. 8 shows the patient interface device of FIG. 7 from the front; and

FIG. 9 shows a third example of device of the invention.

### DETAILED DESCRIPTION OF THE EMBODIMENTS

The invention provides a sensor device in the form of a patient interface, and in which a sensor arrangement is provided for determining parameters relating to the degree of fitting of the contact surface to the patient. This enables design parameters for a customised patient interface to be determined, or enables fitting adjustments to be made based on how well the device fits the patient.

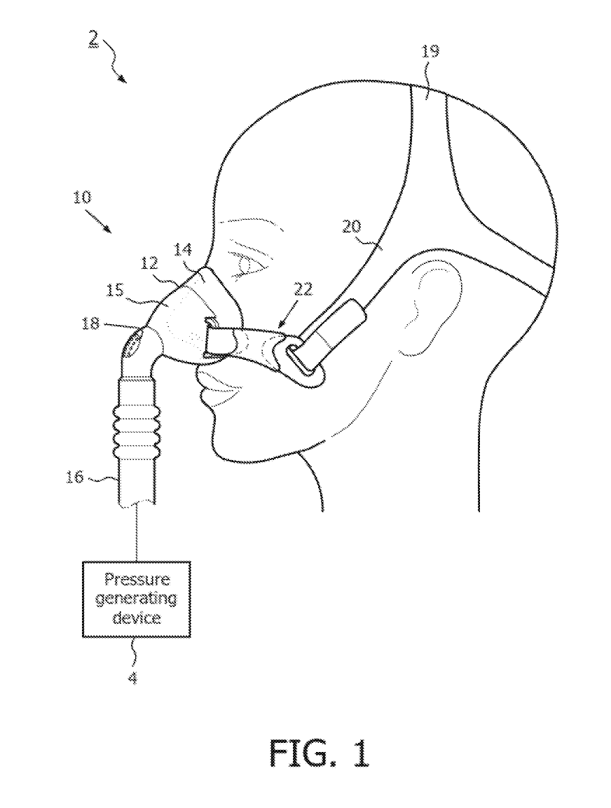

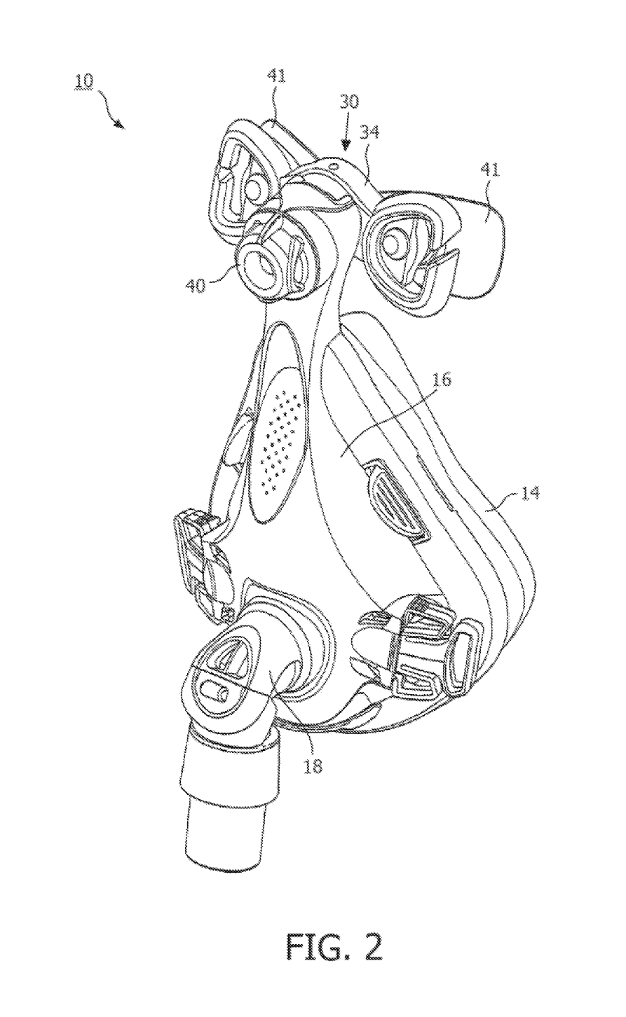

FIG. 2 is taken from U.S. 2010/0000542 and shows a patient interface in the form of a full facial mask assembly 10 including a forehead support 30. The mask part can be considered to comprise a patient interface element, and it is the part for delivering breathing gas to the user. The patient interface element includes a frame 16, a cushion 14 adapted to form a seal with the patient's face, an elbow assembly 18 for connection to an air delivery tube (components 10, 14, 16, 18 corresponding to those of the same number in FIG. 1).

FIG. 2 also shows a forehead support 30 for reducing the forces on the patient's face, and including a frame 34 and forehead cushions 41. In this example, the position of the forehead support is adjustable by a rotary knob 40.

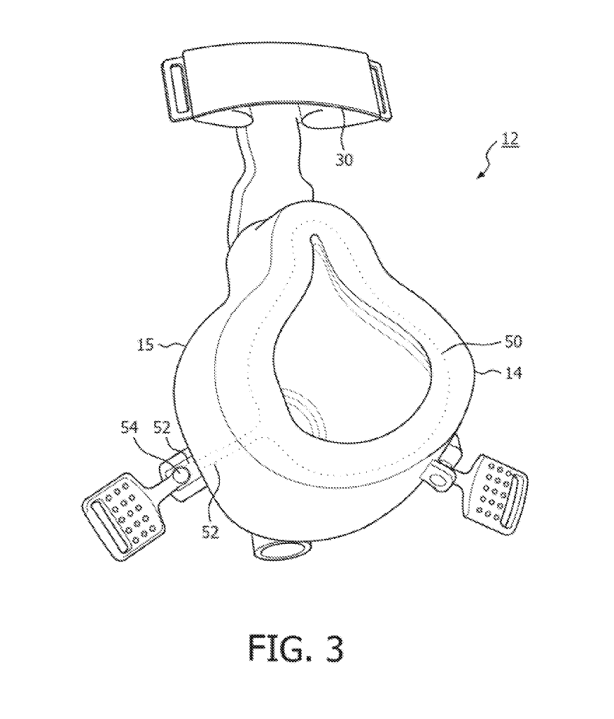

FIG. 3 shows a customisation device 12 of the invention, similar in structure to the design of FIG. 2, with a patient interface comprising a cushion 14 and shell 15 and a forehead support 30.

In this example, the customization is only shown for the patient interface element (the mask part) but it may be provided instead or as well for the forehead support.

Inside or at the surface of the cushion 14, a seal cushion deflection sensor arrangement is integrated or attached.

In the embodiment of FIG. 3, a Fiber Bragg Grating (FBG) sensor 50 is integrated into the cushion 14 to measure the 3D shape of the skin-seal profile when the patient interface element is put on the face. The FBG sensor comprises a fiber ring around the seal area. The sensor is sensitive to strain induced by the deformation of the cushion.

The two ends 52 of the fiber are guided towards a sensing unit 54 on the device housing or as part of a separate analysis device.

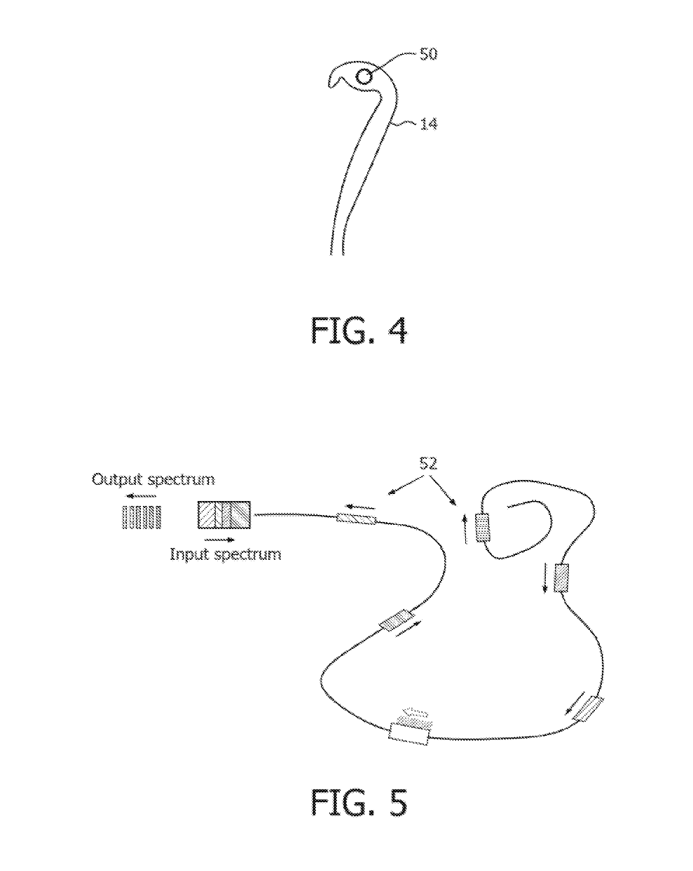

FIG. 4 shows a cross section of the cushion 14 and shows the optical fiber 50 embedded into the cushion material, and running near the surface where contact is made with the patient's face.

The use of a Fiber Bragg grating as a strain sensor is known.

Fibre Bragg gratings (FBG's) enable short sections of optical fibres within fibre optic sensors to be used to detect changes in the local environment around the fibre such as strain, pressure and temperature. The detection of strain can be used as a measure of the contact pressure of the seal surface against the patient's face.

An attractive feature of FBG sensors is the ability to fabricate arrays of sensors at multiple locations along a single fibre, as illustrated in FIG. 5. Each box 52 represents a fiber Bragg grating, each one reflecting a different wavelength.

The different FBG's along the fiber are uniquely identifiable using optical techniques such as wavelength division multiplexing, where, in their quiescent state, the gratings are arranged to reflect different wavelengths back along the optical fibre. The measured quantity such as strain or static pressure can then be determined from the wavelength reflected from each grating.

The FBG's are typically etched on the fibre by UV laser illumination using a phase mask or an interferometer. FBG's have sensing gauge lengths of around 0.1-10 mm and act as a wavelength selective mirror in the core of the fibre. FBG sensors in this form are then primarily temperature and strain dependent. These variables generate changes in the grating period and/or the effective refractive index of the propagating wavelength mode. The resulting changes in the reflected wavelength may subsequently be detected using a spectrometer by interrogating the fibre output using suitable sensors.

From the spectral output, strain and static pressure values are obtained from the fibre and can be translated into deflection values of the mask contour.

The measured quantities are wavelength encoded. This allows demodulation schemes to be used that are insensitive to source power fluctuations and to connector and bend losses. The FBG's are intrinsic to the optical fibre, and an array of FBG's may be readily multiplexed into a single optical fibre to provide multiple measurement points along the fibre with a spatial resolution as high as 0.1 mm (although such high resolution is not needed in this application) and data rates in the order of kilo Hertz (kHz). Long fibre lengths can be encoded at multiple points and interrogated without any significant loss of signal.

This type of fibre optic sensor offers small dimensions (typically, 80-125 μm in diameter), low weight, a large operating temperature range and has highly flexibility structures. A 0.2 mm diameter fibre can for example have a bend radius as low as 2 mm which allows integration in the patient interface element cushion.

The shape of the fiber ring can be determined from the set of strain values and can be used to customize a patient interface element using a manual or automated shaping device. The sensing is carried out as close as possible to the skin contact area to provide a measure of the strain at the skin contact.

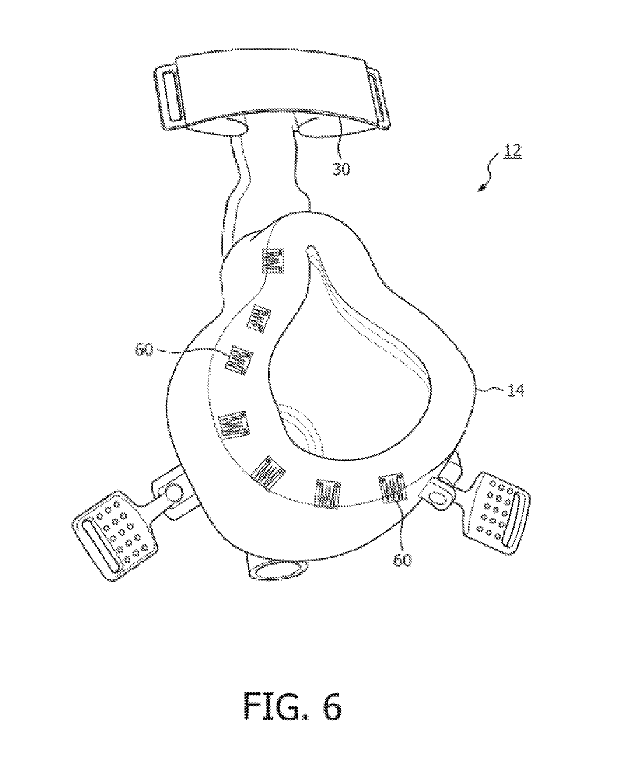

FIG. 6 shows an alternative design, which uses discrete strain gauges 60 which can also be embedded in the patient interface element seal cushion 14, to determine its deformation. The strain gauges can comprise well known metal-foil strain gauges. Alternative designs of strain gauge include conductive pressure sensitive rubbers or electroactive polymers which measure strain as a resistance change with deformation.

In general, the sensor arrangement can comprise a chain (a 1D pattern) or a 2D pattern of sensors.

Instead of using strain measurements, distance measurement may be employed. A distance from a sensor to the skin can indicate if the cushion has been compressed or if there is an air gap to the face.

Distance measurement can for example be based on capacitive sensing, optical or fiber optic or other proximity sensing or ultrasonic probes (for example as used in parking sensors).

The template device with the sensor arrangement can be the one that is then customised by reshaping, or else the template device can be used only for the shape capture, and the information is then used to modify another patient interface, which for example has the same basic design as the template, in particular the same size.

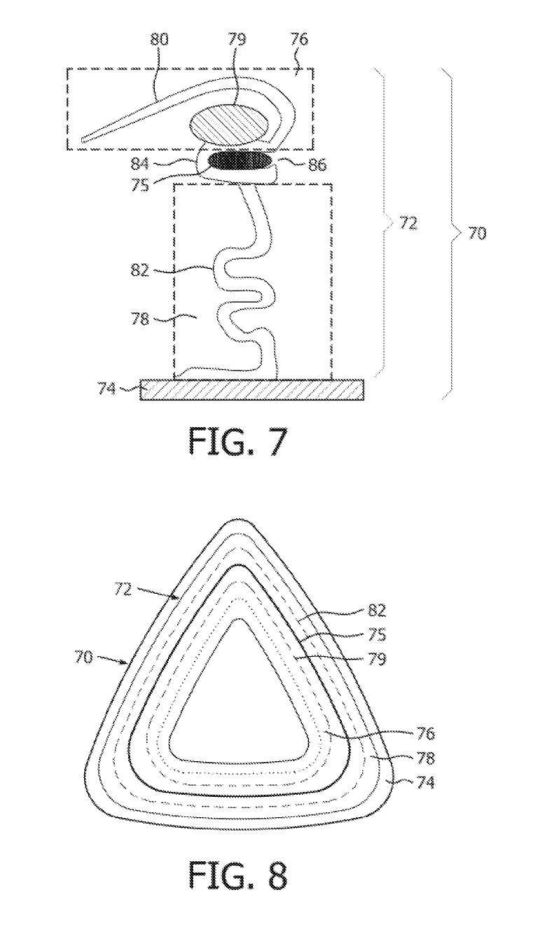

The rehsaping can be carried out in a variety of ways. One example will be explained with reference to FIGS. 7 and 8.

Referring to FIG. 7, a customizable patient interface 70 is illustrated. The patient interface 70 includes a cushion 72 adapted for contacting the user's face, a base 74, a customized element 75, and means for joining the customized element 75 with the cushion 72. The customized element 75 affects at least partially the shape of the cushion 72. Cushion 72 is a flexible structure provided to a substantially rigid or semi-rigid frame member, the base 74, and adapted to engage with the user's face.

The cushion 72 may engage with certain areas of a user's face such as the chin area, the mouth area, the nasal area, the nasal-mouth area, the forehead area or may outline of the entire user interface device.

Cushion 72 includes a facial interface 76 adapted for contacting the user's face and a support interface 78 positioned between the facial interface 76 and the base 74. The facial interface 76 is typically optimized for maximum comfort for the user and support interface 78 is typically optimized for flexibility of the cushion 72. The facial interface 76 includes a core 79 adapted for providing flexibility and strength and is made of a deformable material, such as, for example, a polymer. The facial interface 76 further includes an integrated air tight-flap 80 adapted to engage with the user's face. In alternative embodiments, the air-tight flap 80 can be a separate part. Due to the usage of customized element 75, the facial interface 76 does not have any controlling function for the shape of cushion 72 and, therefore, can be made from a flexible material to be extra soft. The support interface 78 also includes a core 82 adapted for providing flexibility and strength. The core 82 is made of a deformable material, such as, for example, a polymer and can contain spring like elements embedded in such material. Support interface 78 is mechanically connected with the base 74 of the user interface device 70.

Cushion 72 further includes a chamber 84 adapted for receiving the customized element 75. Chamber 84 may be positioned between support interface 78 and facial interface 76 along the periphery of cushion 72. Chamber 84 may include an opening 86 adapted to allow insertion and removal of customized element 75.

Opening 86 of chamber 84 is preferably positioned outside of the breathing path on the outer surface of cushion 72, as shown in FIG. 7, to secure customized element 75 inside chamber 84 when the user interface device expands under the air pressure provided by a respiratory ventilation system. Furthermore, by positioning opening 86 of chamber 84 on the outer surface of cushion 82 and, therefore, outside of the breathing path, customized element 75 may be prevented from contact with the breathing volume inside the user interface device allowing a more flexible design of element 75.

Customized element 75 comprises a pre-formed rigid or semi-rigid structure adapted for corresponding to the shape of the user's face and adapted for extending at least partially along a contour of the user interface device 70. The rigid structure has no direct contact with the user's face and the gas. The shape of the structure is based on the user specific data set obtained in the manner explained above, which for example can be interpreted to give a three-dimensional shape of the user's face. In one embodiment, the shape of the structure is not changeable after being first pre-formed according to the shape of the user's face.

Customized element 75 can be fabricated independently and separately from the rest of the patient interface 70 and may be positioned within chamber 84 at a certain distance from the integrated air-tight flap 80 as shown in FIG. 7. The customized element 75 can be placed at variable distance from the user's face. The distance to the user's face and, thus, the skin surface can be larger in facial areas with extra thin and sensitive skin.

Customized element 75 is relatively rigid or semi-rigid and is responsible for the optimal pressure distribution at the facial interface 76 of the user interface device 70. Customized element 75 is adapted to pre-deform cushion 72, and specifically the facial interface 76, making it compliant with a given face of a particular user. Customized element 75 is a custom fabricated element, where the shape is adapted to match a user specific data set.

Customized element 75 may be fabricated from a metallic spring material or preferably plastic using, for example, a custom pressing. Alternatively, customized element 75 may be made using a rapid prototyping technique such as NC milling or any plastic or metal layered manufacturing technique such as 3D printing, stereo lithography (SLA),

Selective Laser Sintering (SLS), Fused Deposition Modelling (FDM), foil-based techniques, etc. Since customized element 75 does not have contact with the skin of a user, it may be produced from a broad range of materials. Customized element 75 may be made from a 3D printable material, for example, from a relatively strong nylon material having a relatively good heat resistance, such as Nylon 12 or Polyamide PA 2200 using selective-laser-sintering (SLS). Nylon 12 and Polyamide PA 2200, for example, are common materials used in SLS and parts made of these materials have good long term stability, offering resistance to most chemicals. These materials are harmless to the environment and safe to use with food articles. Complexity is irrelevant and the materials deliver the impact strength and durability required for functionality. Tensile and flexural strength combine to make tough plastic prototypes, with the flex associated with many production thermoplastics. It is able to emulate living hinge designs, certainly to 20+ cycles. These plastic materials are non-hygroscopic, thereby negating the requirement to seal the surface on components being used with liquids.

In one embodiment, cushion 72 (excluding part 75) is a pre-fabricated standard article. For example, cushion 72 can be a typical standard cushion adapted for use with known patient interfaces.

Referring to FIG. 8, a schematic top view of the patient interface 70 is illustrated. As can be seen, the customized element 75 extends along the contour of the interface device 70, and specifically, of the cushion 72. The customized element 75 can be formed as a single part adapted to have the shape of a ring. The cross-section of the ring can be elongated in the direction tangent to the user's face to control the local shape of the facial interface 76 and provide better comfort.

The example above requires separate fabrication or shaping of the part 75, which is then applied to a standard template. In this case, the template device used for determining the facial shape can be separate to the one to be customized.

However, in other examples, the template device used for determining the facial shape can be the one to be customized and worn by the patient. The customization of the interface can in one example be made in real time, continuously while the user wears the mask. Thus, muscle relaxation and cushion influences caused by patients sleeping on their side can be taken in account. This requires a customization approach based on actuators which form part of the device. For example, small motors can be used to implement adjustments.

The sensing mechanism and the actuation mechanism for the customization may be integrated. For example, if a motor is used as actuator, the force of the actuation on the skin can be measured as a function of the voltage and current characteristics of the motor.

As will be seen from the above, the invention can be implemented in the cushion part. A cushion part alone with the sensor arrangement can be provided, for attachment to a standard support structure for application to the user during the customisation process. For example, a set of different cushion sizes can be provided which all fit to one standard support structure. In this way, the smallest number of components is varied to enable the customisation to be possible for the largest range of sizes of different users. Different cushions can have different sensing mechanisms.

In the examples above, a single optical fiber ring with distributed fiber Bragg gratings has been proposed. An alternative uses multiple optical fibers, to enable force/pressure vectors to be determined. This type of arrangement has been proposed for determining the deformation of a catheter in WO2006/092707.

In the catheter tip, the use of at least two optical fiber sensors is required to be able to compute at least a two-dimentional force vector. More preferably the tip comprises three optical fiber sensors disposed within the deformable body so that they are not co-planar. This permits the computation of a three-dimensional force vector.

The use of multiple fibers can increase the accuracy of the measurement. Processing logic can be based on a matrix of values associated with the physical properties of an individual deformable body of the mask. More preferably, a force-strain, convertion matrix specific for each deformable body is determined during manufacture and stored on appropriate memory.

Instead of fiber Bragg gratings, long period fiber gratings can be used. Other means of sensing contact forces which can be used include mechanical, capacitive, inductive, and resistive pressure sensing devices.

The examples above are based on obtaining customisation information. It is mentioned above that the customization of the patient interface can be made in real time, continuously while the user wears the mask. This can comprise fitting adjustments (rather than interface shape adjustments) to be made.

These fitting adjustments can be made using the strap arrangement 20 which is for holding the patient interface 14,15 in contact with the face of the patient.

The fit and mask-skin contact pressure of a non-invasive ventilation mask is critical to prevent mask induced skin irritation, sore, and air leakage. Especially the skin of the nose is susceptible for mask induced irritations and sore. The mask comfort is also important for treatment adherence.

This example uses the measurement of the degree of fitting, such as the mask-skin contact pressure, to enable adjustments to be made.

In a most simple version, a warning is provied to the user so that they can make strap adjustments. FIG. 1 shows only one adjustment point for the strap harness, but there may be many. A warning can thus be provided to the patient in relation to a particular strap that is adjusted too tight or too loose and which would thus cause skin irritation in prolonged mask use (if too tight) or else would cause an air leak (if too loose). The patient can then loosen or tighten the particular strap until the measured mask-skin contact pressure is within acceptable limits, which may be patient specific.

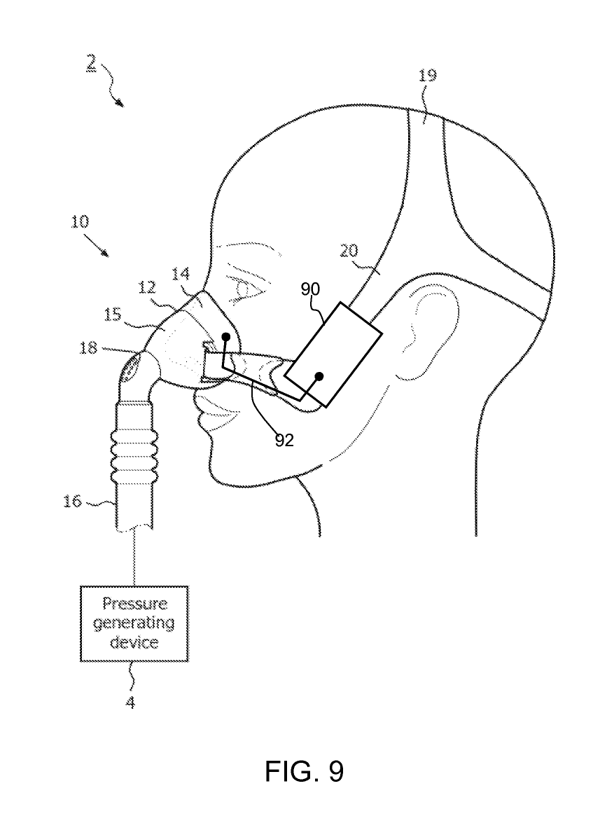

An alternaitve approach is shown schematically in FIG. 9, which has an adjustment means 90 for adjusting the strap arrangement 20. The adjustments are made based on the sensor arrangement signals coming from the cushion 14 (as shown by signal path 92). This provides automatic strap length adjustment based on the measured fitting parameters (such as mask-skin contact pressure) by means of strap integrated actuators.

The actuator 90 automatically adjusts the strap length for exmaple by winding the strap end on a rotating, miniaturized motor driven coil. The adjustment can be done in a hybrid manual-automatic combination mode, to allow larger (one time) adjustments to a patient's head geometry manually, and to allow automatic fine adjustments which are controlled by the sensor readings in a closed-loop control manner.

The inveniton can be used for treatment masks as explained above, but also for ventiallation masks or other breathing masks e.g. for personal protective equipment (e.g. gas masks, dust/particle filtering masks, breathing apparatus for firefighters, etc.).

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. Any reference signs in the claims should not be construed as limiting the scope.

1. A sensor device comprising:
a patient interface cushion having a contact surface for making contact with the face of the patient when the sensor device is worn by the patient; and
a sensor arrangement provided for determining a parameter relating to the degree of fitting of the contact surface to the patient, thereby to enable design parameters to be obtained for a customized patient interface or to enable fitting adjustments of the patient interface to be made.

2. A device as claimed in claim 1, wherein the sensor arrangement is provided inside or at the contact surface of the cushion, for determining a degree of fitting of the contact surface to the patient.

3. A device as claimed in claim 1, wherein the patient interface is for communicating a breathing gas to a patient.

4. A device as claimed in claim 1, wherein the patient interface comprises a shell and the cushion, and wherein the cushion is provided with the sensor arrangement.

5. A device as claimed in claim 1, wherein the sensor arrangement comprises: an optical fiber providing strain, pressure, force or distance information at different positions around the contact surface; or an array of strain gauges around the contact surface.

6. A device as claimed in claim 1, wherein the patient interface comprises a patient interface element and a forehead support, wherein the sensor arrangement is for determining a degree of fitting of the contact surface of either one or both of the patient interface element and the forehead support to the patient.

7. (canceled)

8. A customization system for a patient interface, comprising:
(a) a customization sensor device comprising:
(1) a patient interface cushion having a contact surface for making contact with the face of the patient when the sensor device is worn by the patient, and
(2) a sensor arrangement provided for determining a parameter relating to the degree of fitting of the contact surface to the patient, thereby to enable design parameters to be obtained for a customized patient interface, or to enable fitting adjustments of the patient interface to be made; and
b) a processor for determining, from an output of the sensor arrangement, design parameters for a patient interface.

9. A customization system as claimed in claim 8 which comprises a set of customization sensor devices, further comprising a set of patient interfaces of different sizes corresponding to the sizes of the customization sensor devices, wherein each patient interface has a contact surface for making contact with the face of the patient when the device is worn by the patient, wherein the shape of the contact surface is adjustable.

10-11. (canceled)

12. A method of customizing a patient interface, comprising:
applying a sensor device to a patient, the sensor device comprising a patient interface having a contact surface for making contact with the face of the patient when the device is worn by the patient and a sensor arrangement for determining a parameter relating to the degree of fitting of the contact surface to the patient, wherein the sensor arrangement is provided inside or at the contact surface of the cushion;
from the parameter relating to the degree of fitting of the contact surface to the patient obtaining design parameters for a customized patient interface; and.

13. A method as claimed in claim 12, comprising a method of customizing a patient interface, wherein the method comprises:
determining from the sensor arrangement signals design parameters for a customized patient interface; and
customizing a patient interface using the design parameters.

14. A method as claimed in claim 13, comprising:
selecting one customization sensor device from a set of customization sensor devices of different sizes, wherein the selected customization device is the one applied to the patient; and
selecting one of a set of patient interfaces of different sizes, the one selected having a corresponding size to the selected customization sensor device, and wherein the design parameters are used to customize the selected patient interface.

15. (canceled)
